(19) United States
(12) Patent Application Publication
(10) Pub. No.: US 2012/0141788 A1
(43) Pub. Date: Jun. 7, 2012

(54) PROCESS AND DEVICE FOR THE PRECIPITATION OF AN ORGANIC COMPOUND

(75) Inventor: Huibert Albertus Van Boxtel, Tilburg (NL)

(73) Assignee: Fujifilm Manufacturing Europe B.V.

(21) Appl. No.: 12/441,884

(22) PCT Filed: Sep. 19, 2007
(86) PCT No.: PCT/NL2007/000233

§ 371 Date: Oct. 28, 2009

(30) Foreign Application Priority Data
Sep. 19, 2006 (EP) 06120915.1
Jul. 20, 2007 (GB) PCT/GB2007/002786

(51) Int. Cl. B32B 5/16; C07J 7/00; B01D 21/01; C07D 305/14; C07K 7/64; B01D 9/02; C07C 69/738

(52) U.S. Cl. 428/402; 552/606; 560/52; 549/510; 530/321; 210/198.1

(57) ABSTRACT

The present invention relates a process for the precipitation of an organic compound, wherein:
(a) a solution (I) of the organic compound in a first solvent is introduced via a first inlet into a closed type mixing chamber;
(b) a precipitation agent (II) is introduced, simultaneously with step (a), via a second inlet into the closed type of mixing chamber;
(c) the solution (I) of the organic compound and the precipitating agent (II) are mixed thereby forming a precipitate of the organic compound and a liquid phase; and
(d) discharging the precipitate of the organic compound and the liquid phase via a single outlet from the closed type mixing chamber.

### FIELD OF THE INVENTION

The present invention relates to a process and a device for the precipitation of organic compounds and derivatives thereof, e.g. precursors, (addition) salts, polymorphs, solvates and hydrates of these organic compounds. The organic compounds may be amorphous or crystalline organic compounds. The organic compounds are in particular for use as a pharmaceutical active agent for the treatment or the prophylaxis of a disease or discomfort. However, the organic compound may also be a precursor of a pharmaceutically active agent. Additionally, within the scope of this invention, the organic compound may also be a valuable excipient of a pharmaceutical formulation comprising a pharmaceutical active agent.

### BACKGROUND OF THE INVENTION

Crystallization from solution of pharmaceutically active compounds or their intermediates is a typical method of purification in the industry. It is very important to obtain the desired crystal average size, size distribution, morphology, polymorph and purity of the active ingredient. In the case of drugs that are slightly soluble in water, e.g. those having a solubility less than 10 mg/ml, the crystal size strongly affects the dissolution rate and equilibrium solubility, i.e. that a smaller crystal size results in a higher dissolution rate and equilibrium solubility which is desired. These factors reflect the drug bioavailability in the human body. The direct crystallization of small sized high surface area particles is usually accomplished in a high supersaturation environment which often results in material of low purity, high friability, and decreased stability due to poor crystal structure formation.

Poorly water-soluble drugs also tend to be eliminated from the gastro-intestinal tract before being absorbed into the circulation and too large particles, e.g. a diameter of more than 6 μm can give rise to difficulties when required for intraveneous administration in terms of blocking needles and even blocking tiny blood vessels, e.g. capillaries, in patients.

Slow crystallization is a common technique used to increase product purity and produces a more stable crystal structure, but it is a process that decreases crystallizer productivity and often produces large, low surface area particles that require subsequent high energy milling. Currently, pharmaceutical compounds almost always require a post-crystallization milling step to increase particle surface area and thereby improve their bioavailability. However, high energy milling has drawbacks. Milling may result in excessive local temperatures resulting in degradation of material, yield loss, noise and dusting, as well as unwanted personnel exposure to highly potent pharmaceutical compounds. Additionally, in conventional dry milling, the limit of fineness is usually about 100 μm when material begins to cake on the walls of the milling chamber. Although wet milling is suitable in further reducing fineness, flocculation usually restricts the lower particle size to about 10 μm. Air jet milling techniques may provide average particle sizes of about 1 to about 50 μm. Also, stresses generated on crystal surfaces during milling can adversely affect labile compounds. Overall, the three most desirable end-product goals of high surface area, high chemical purity, and high stability are notoriously difficult to optimize simultaneously using current crystallization technology without high energy milling.

In order to get micron-sized or nano-sized crystals a high supersaturation should be used. The standard problem using high supersaturation is that it results into an extremely rapid nucleation rate and subsequently a high growth rate. The design of a crystallization method and a crystallisation apparatus to achieve the minimum necessary rate of mixing in the case of high supersaturation has been notoriously difficult. Estimated nucleation rates of milli- or even microseconds are not uncommon for solvent/anti-solvent and for reaction precipitations. Homogeneous nucleation rates can be estimated using classical nucleation theory, see Kashchiev, Nucleation, Basic Theory with Applications, Butterworth-Heinemann, 2000 and Kashchiev and van Rosmalen Review Nucleation in solutions revisited, Cryst. Res. Technol., vol 38, No. 7-8, 555-574, 2003, taking into account improved estimates for the particle-solution interfacial energy, see Granberg et al, Primary nucleation of paracetamol in acetone-water mixtures, Chem. Eng. Sci., vol 56, 2305-2313, 2001. In case of solvent anti-solvent precipitation supersaturation can become high, especially when applying the so-called “reverse” addition sequence. Reverse addition means adding the solute solution to the anti-solvent. This typically creates a higher supersaturation than adding the anti-solvent to the solute solution. A supersaturation ratio of 10 or more is considered high in this context.

There has been extensive research and development in the field of the precipitation or crystallisation of organic compounds, in particular in the field of mixing two reacting solutions quickly and completely to cause precipitation of a solute into micro- or nano-sized particles with a narrow particle size distribution. The mixing process should preferably be finished, that is, in the mixing process the two solutions should have mixed into a molecularly dispersed homogeneous mixture, before the nucleation of particles starts. Any inhomogeneities will usually broaden the particle size distribution. In case particles are desired with an average size roughly less than 1 μm, usually stabilisation by a proper additive is preferred. Particles of such a small size are typically formed under conditions of high to very high supersaturation, the condition under which particle growth is also very rapid. The particle growth, however, should be low to allow the creation of many particles (nucleation) to continue for a longer time before the supersaturation has diminished to negligible values and nucleation stops. Such additives are compounds that adsorb to the crystal surfaces and block crystal surface growth sites. Suitable additives are typically amphiphilic polymers or copolymers, surfactants or any combination thereof.

The standard equipment for crystallization is the Continuously Stirred Tank Reactor (CSTR). It is provided with a stirrer for mixing and baffles to limit the creation of a vortex whenever rapid stirring speeds are necessary. There are several disadvantages of this type of crystallizer including the inhomogeneous nature of the stirring power, causing inhomogeneous mixing, foaming and particle agglomeration, to name a few. A small particle size with a narrow particle size distribution cannot be obtained in general using this method.

Another crystallisation method makes use of an impinging jet as disclosed in EP A 1,157,726 and U.S. Pat. No. 5,314,506, and in Chapter 18 of Johnson et al., ACS Symposium series (2006), 924 (Polymeric Drug Delivery II), 278-291 (published by the American Chemical Society, CODEN: ACSMC8 ISSN: 0097-6156. Here, jet streams are directly impinged to create high turbulence at the point of impact under conditions of temperature and pressure that permit micro mixing of the solutions. The impinging jet method generally comprises providing two substantially diametrically opposed jet streams that impinge to create an immediate high turbulence impact. One standard crystallization process involves mixing a supersaturated solution of the compound to be crystallized with an appropriate “anti-solvent” or crystallizing agent. Another standard crystallization process employs temperature variation of a solution of the compound to be crystallized in order to bring the solution to its supersaturation point.

With the known opposed impinging jet methods, it is possible to prepare crystalline products with particles sized between 5 to 1000 μm. A disadvantage of the opposed impinging jet method is the accuracy of the positioning and alignment of the jet nozzles, because if the jets are only slightly out of line, the solution and anti-solvent will not mix sufficiently resulting in a wider particle size distribution. In case of small deviations, the jet nozzles might also get clogged by crystallisation at the nozzle. Furthermore, insufficient flow rates from one or more of the jet nozzles may affect the quality of the entire batch being produced, especially if a majority of the solutions are not micro mixed at the desired point of impact. In such a case a narrow small size particle distribution cannot be achieved. Generally, the preferred flow for the impinging jet streams has little room for variance.

EP A 1,157,726 and U.S. Pat. No. 6,302,958 further disclose the use of an impinging jet device with a sonication probe to achieve high intensity mixing. This method has similar disadvantages as the impinging jet method.

US 2005/202095 (WO 03/033097) discloses a high intensity, in-line rotor-stator apparatus for the production of small particles via anti-solvent, reactive, salting out or rapid cooling precipitation and crystallisation. However, the apparatus disclosed in US 2005/202095 has various disadvantages. First of all, oversaturation is rather difficult to control. Co-introduction of solvents and anti-solvents at the same position (cf. FIG. 1, reference numbers 9 and 10) may lead to unstable mixtures. Additionally, the apparatus has dead spaces where mixing hardly occurs which results in inhomogeneous mixtures and broad particle size distributions. More importantly within the context of the present invention, the device disclosed in US 2005/202095 comprises a “closed mixing type chamber” defined as “first cavity 3” that encloses a stator 5, said stator 5 being provided with a multitude of “outlets” defined as apertures 13. In addition, the “first cavity 3” comprises “volutes 14”, which form an annular gap between the outer wall of the “closed mixing type chamber” defined as “first cavity 3” and the stator 5. In these “volutes 14”, mixing hardly occurs so that for a person skilled in the art the “volutes 14” are not a part of a “closed mixing type chamber”. Consequently, the “closed mixing type chamber” according to US 2005/202095 is provided with a multitude of outlets defined as apertures 13. In addition, large particles are produced, as the Examples indicate that the precipitated glycine particles ranged in diameter between about 4.4 to 300 μm.

In the field of precipitation of inorganic salts U.S. Pat. No. 5,985,535 discloses a process for producing silver halide emulsions comprising a specific precipitation step that is performed in a particular apparatus. The apparatus comprises a closed type stirring tank provided with stirring means positioned essentially opposite to each other. The stirring means are driven at high speed, i.e. 1000 rpm or more, in converse directions. The stirring means are also driven without a rotary axis protruding through the closed type stirring tank, but by employing a magnetic coupling with an outer magnet that is connected to a motor. The suitability of this apparatus for the precipitation of organic substances is not disclosed.

Because the methods and devices as described above for the crystallization of organic compounds have several disadvantages, there is a need for a method and a device providing the creation of organic/pharmaceutical particles with a small average size and a reproducible and narrow average size distribution having a high purity.

### SUMMARY OF THE INVENTION

In their search for efficient and reproducible methods for the precipitation of organic/pharmaceutically active compounds into very small particles with a narrow particle size distribution, the present inventors designed a method and a device for the controlled continuous precipitation of an organic compound in a closed type mixing chamber where, after an induction period, an in time stable mixture of a precipitate and a liquid phase is obtained in the closed type mixing chamber. Accordingly, the present invention relates therefore to a process for the precipitation of an organic compound, wherein:
(a) a solution (I) of the organic compound in a first solvent is introduced via a first inlet into a closed type mixing chamber;
(b) a precipitation agent (II) is introduced, simultaneously with step (a), via a second inlet into the closed type of mixing chamber;
(c) the solution (I) of the organic compound and the precipitating agent (II) are mixed by sonication, by a rotatable magnetic stirring means or by a rotatable mechanical stirring means thereby forming a precipitate of the organic compound and a liquid phase; and
(d) the precipitate of the organic compound and the liquid phase is discharged from the closed type mixing chamber via a single outlet.

The present invention further relates to a device 1 for the precipitation of an organic compound, wherein the device comprises:
(a) a closed type mixing chamber 3;
(b) an inlet 4 for feeding a solution (I) of the organic compound in a solvent, the inlet 4 being connected to the closed type mixing chamber 3;
(c) an inlet 5 for feeding a precipitating agent (II) to the closed type mixing chamber 3, the inlet 5 being connected to the closed type mixing chamber 3;
(d) a single outlet 6 for receiving a precipitate of the organic compound and a liquid phase, the outlet 6 being connected to the closed type mixing chamber 3; and
(e) a mechanical stirring means 2, wherein the mechanical stirring means 2 is comprised by the closed type mixing chamber 3.

Hence, in such a closed type mixing chamber in which effective mixing is achieved essentially in the whole volume of the closed type mixing chamber, a single outlet provides distinctive advantages over the apparatus disclosed in US 2005/020295.

Additionally, the present invention relates to the use of a precipitate obtainable by the process and/or the device of the invention as an active pharmaceutical ingredient in a pharmaceutical composition or formulation and to the use of a precipitate obtainable by the process of the invention for the manufacture of a pharmaceutical composition or formulation, i.e. as an excipient or additive or as a pharmaceutical active component.

### BRIEF DESCRIPTION OF THE DRAWINGS

FIG. 1 shows a general representation of the device of the present invention.

FIG. 2 shows a cross-sectional view of a preferred embodiment of the device of the present invention.

FIG. 3 shows a cross-sectional view of another preferred embodiment of the device of the present invention.

FIGS. 3A and 3B show top views of a more preferred embodiment of the device shown in FIG. 3.

FIG. 4 shows a cross-sectional view of yet another preferred embodiment of the device of the present invention.

FIG. 5 shows crystals of pregnenolone obtained in a crystallisation process that is performed in a conventional continuous stirred tank reactor (CSTR).

FIG. 6 shows the particle size distribution of the crystals of pregnenolone shown in FIG. 5.

FIG. 7 shows crystals of pregnenolone obtained in a crystallisation process that is performed in a device according to FIG. 2.

FIG. 8 shows the particle size distribution of the crystals of pregnenolone shown in FIG. 7.

### KEY TO THE SYMBOLS

1: Precipitation device
2, 2a, 2b: Stirring means
3: Closed type mixing chamber
4: First inlet for feeding a solution (I)
5: Second inlet for feeding a precipitating agent (II)
6: Outlet for receiving a precipitate of the organic compound and a
liquid phase
7: Tank body
8: Seal plate
9, 9a, 9b: Stirring blade
10, 10a, 10b: Outer magnet
11, 11a, 11b: Motors
12: Axis or shaft
13: Moveable chamber part
14: Hinge
15: Stirring disk
16: Mixing zone
17: Separating wall.

### DETAILED DESCRIPTION OF THE INVENTION

In this document and in its claims, the verb “to comprise” and its conjugations is used in its non-limiting sense to mean that items following the word are included, but items not specifically mentioned are not excluded. In addition, reference to an element by the indefinite article “a” or “an” does not exclude the possibility that more than one of the elements is present, unless the context clearly requires that there be one and only one of the elements. The indefinite article “a” or “an” thus usually means “at least one”.

In this document, the term “organic compounds” in its broadest sense refers to compounds comprising at least one carbon atom. Usually, organic compounds also comprise hydrogen atoms. Very often organic compounds also comprise hetero-atoms, e.g. oxygen atoms, nitrogen atoms, and/or sulphur atoms. In particular the term “organic compounds” refers what is normally considered an organic compound in the field of pharmaceutical, dye, agricultural and chemical industry. The term “organic compounds” also include compounds that comprise a metal atom, i.e. organometallic compounds such as haemoglobin, and salts. The term “organic compounds” includes “biological” organic compounds such as hormones, proteins, peptides, carbohydrates, amino acids, lipids, vitamins, enzymes and the like. The term “organic compounds” also encompasses different crystalline forms, i.e. polymorphs, hydrates and solvates, as well as salts including addition salts.

The term “precipitation” refers to a subclass of the field of solution precipitation. Precipitation is often recognised by one or more of the following characteristics: (i) low solubility of the precipitated particles, (ii) fast process, (iii) small particle size and (iv) irreversibility of the process (W. Gerhartz in: Ullmans encyclopedia of Industrial Chemistry, vol. B2 5^{th} ed., VHC Verlagsgessellschaft mbH, Weinheim, FGR, 1988). In the context of this invention, a suitable definition for precipitation is the relatively rapid formation of a sparingly soluble solid phase from a liquid solution phase (Handbook of Industrial crystallization, Edited by Allan S. Myerson, Butterworth Heinemann, Oxford, p141).

Generally two types of processes resulting in precipitation can be discerned:
a first type of such a process is anti-solvent (also referred to as non-solvent) precipitation. A dissolved organic compound is mixed with a solvent that lowers its solubility so that a precipitate will form. A modification of the anti-solvent precipitation is that a dissolved organic compound is not necessarily mixed with an anti-solvent but is mixed in such way that the solubility of the precipitating solvent is lowered such that nuclei are formed. This can be realised by variations in for example temperature, pH (addition of acid or alkaline solutions), ionic strength and the like and combinations of such factors.
a second type of such a process is reaction precipitation. Two components are mixed resulting in the formation of a newly formed organic compound and due to the low solubility of the formed organic compound under the used mixing or reaction conditions a precipitate will form.

Obviously, the term “precipitation” encompasses any process wherein small solid particles are formed, e.g. including but not limiting to crystallisation.

The term “anti-solvent” or “non-solvent” is normally to be understood as a solvent in which the solubility of the organic compound is less than 1% by weight, preferably less than 10⁻²% by weight, based on the total weight of the solvent and the organic compound, at a temperature of 20° C. and a pressure of 1 bar. The solvent may be polar or apolar. The solvent may be protic or aprotic. The solvent may further be non-ionic or ionic. However, under certain conditions, the solvent may also be a gas in the supercritical state, e.g. supercritical carbon dioxide. Obviously, the solvent may be a mixture of different solvents, wherein the mixture is either essentially homogeneous or comprises two phases. However, it is preferred that the solvent comprises an organic solvent. It is even more preferred that the solvent is an organic solvent. Additionally, it is preferred that the solvent used for forming the solution of the organic compound is miscible with the anti-solvent.

With the term “over-saturation” (or “supersaturation”) is meant a concentration of an organic compound that is in excess of saturation under the given conditions, i.e. solvent or solvent mixture, temperature, pH, ionic strength etc.

In the process according to the present invention, a solution (I) of the organic compound or a precursor of the organic compound in a first solvent is provided which is fed with a continuous flow via a first inlet into the closed type mixing chamber. Simultaneously, a precipitation-agent (II) is fed, also with a continuous flow, via a second inlet into the closed type mixing chamber. The closed type mixing chamber may be provided with more than one first inlet for this solution (I) and more than one second inlet for this precipitating agent (II). In a next step, the solution (I) and the precipitation agent (II) are mixed and said mixture provides an oversaturation, preferably a stable over-saturation S10, of the organic compound resulting in the formation of a precipitate of the organic compound and a liquid phase. The term “S10” indicates the oversaturation ratio S at 10 seconds after the start of addition. It is defined as:

$${S}_{10} = \frac{{C}_{10}}{{C}_{10 , e}} ,$$

in which C₁₀ equals the calculated concentration of solute at 10 seconds after start of addition, C_{10,e} equals the equilibrium solute concentration of solute at 10 seconds after start of addition

Finally, the mixture of the precipitate and the liquid phase is discharged from the closed type mixing chamber, preferably also with a continuous flow, and preferably into a collecting (or receiving) vessel. According to the invention, it is preferred that there is basically no oversaturation at the single outlet of the closed type mixing chamber. Additionally, according to the invention, there are no other openings in the closed type mixing chamber besides the inlets and the single outlet. This means that no solvents, liquids, solutions, particles and the like can enter or exit the closed type mixing chamber except via the first and second inlets and the single outlet.

The solution (I) of the organic compound may comprise a single solvent or a mixture of solvents, wherein the solvent or solvents may be polar or apolar, protic or aprotic, and/or non-ionic or ionic. The solvent may also be a gas in the supercritical state, e.g. supercritical carbon dioxide, if that is appropriate.

The preferred nature and composition of the precipitation agent (II) is dependent on the organic compound and the process used and can for example be a solution having a lower temperature (in case of low temperature precipitation), different ionic strength or different pH than the solution (I). The precipitating agent (II) can also be a non-solvent, a mixture of non-solvents, or a mixture of a non-solvent and a solvent.

The process according to the present invention is very suitable for the preparation of very small particles with a narrow average particle size distribution in the lower micron, sub-micron or even nanometer range. A disadvantage of such small particles is that these tend to be unstable; therefore stabilisation agents and/or wetting agents are preferably added to increase the stability of these particles. Accordingly, it is preferred that the solution (I) and/or the precipitating agent (II) comprises a stabilising agent. It is furthermore preferred that the solution (I) and/or the precipitating agent (II) comprises a wetting agent.

According to the invention, these stabilisation agents preferably comprise a component which is selected from the group consisting of a protein, an enzyme, a peptide, a polypeptide, a gelatine, an amino acid, an amphiphilic polymer, a nucleotide, an oligonucleotide, a RNA sequence, a DNA sequence, a carbohydrate, a polysaccharide, an oligosaccharide, a disaccharide, a monosaccharide, a lipid, a fatty acid, wherein the fatty acid may be saturated, unsaturated or multiply unsaturated, a phytochemical, a vitamin, a mineral, a salt, a colorant, a pigment, a sweetener, an anti-caking agent, a thickener, an emulsifier, an anti-microbial agent, an antioxidant, and mixtures thereof. The stabilising agent preferably comprises an amphiphilic polymer.

More preferably, the stabilising agents are selected from the group consisting of a protein, an enzyme, a peptide, a polypeptide, a gelatine, an amino acid, an amphiphilic polymer, a nucleotide, an oligonucleotide, a RNA sequence, a DNA sequence, a carbohydrate, a polysaccharide, an oligosaccharide, a disaccharide, a monosaccharide, a lipid, a fatty acid, wherein the fatty acid may be saturated, unsaturated or multiply unsaturated, a phytochemical, a vitamin, a mineral, a salt, a colorant, a pigment, a sweetener, an anti-caking agent, a thickener, an emulsifier, an anti-microbial agent, an antioxidant, and mixtures thereof. The stabilising agent preferably is an amphiphilic polymer.

Preferred amphiphilic polymers are amphiphilic block polymers, more preferably amphiphilic block copolymers. The preferred amphiphilic block copolymers are selected from the biocompatible amphiphilic block copolymers for which the preferred block-types and block-lengths can vary depending on the organic compound to be precipitated and on the preferred average particle size after precipitation.

Preferably, the amphiphilic block copolymer comprises hydrophilic and hydrophobic blocks. Additionally, it is preferred that the amphiphilic block copolymer is a diblock copolymer or a triblock copolymer, more preferably a diblock copolymer, in particular a diblock copolymer having a hydrophilic block and a hydrophobic block.

Preferred hydrophilic blocks are poly(ethylene glycol) (“PEG”) and/or poly(ethylene glycol) monoether (“PEG ether”) blocks. The preferred ethers have 1 to 4 carbon atoms, preferably 1 carbon atom, and most preferably the ether is a methyl ether.

Preferred hydrophobic blocks are poly(lactic-co-glycolic)acid (“PLGA”), poly(styrene), poly(butyl acrylate), poly(ε-caprolactone) and in particular polylactide (“PLA”) blocks. Polylactides are polyesters formed by polymerisation of dilactide, i.e. the dimer of lactic acid and may occur in different stereochemical configurations, e.g. poly-L-lactide, poly-D-lactide and poly-L,D-lactide.

Preferred biocompatible amphiphilic block copolymers include copolymers comprising a PEG and/or a PEG ether block and a PLA block.

Preferably, the PEG and PEG ether blocks have a Mₙ of 250 to 5000, more preferably 400 to 4000, even more preferably 500 to 2000. In a preferred process of the present invention, the amphiphilic polymer is an amphiphilic block copolymer comprising a PEG block having a Mₙ of 250 to 5000 and/or a PEG (C₁-C₄ alkyl)ether block having a Mₙ of 250 to 5000, wherein the Mₙ of the PEG block or the PEG (C₁-C₄ alkyl)ether block is more preferably 400 to 4000, even more preferably 500 to 2000.

The PLA block has preferably a Mₙ of 250 to 5000, more preferably 400 to 4000, even more preferably 500 to 2000. Good results were obtained for example with a PLA block having a Mₙ of 1000.

A particularly preferred biocompatible amphiphilic block copolymer is a diblock copolymer of a PEG (C₁-C₄ alkyl)ether block and a PLA block, wherein the PEG (C₁-C₄ alkyl)ether block and PLA block have a Mₙ as mentioned above.

Preferred biocompatible amphiphilic diblock copolymers include poly(ethylene glycol)-PLA diblock copolymers, in particular:

Poly(ethylene glycol)-block-polylactide (C₁-C₄ alkyl)ether, PEG Mₙ 350 to 1500 (preferably 750), PLA Mₙ 500 to 2000 (preferably) 1000;
Poly(ethylene glycol)-block-polylactide (C₁-C₄ alkyl)ether, PEG Mₙ 350 to 1500 (preferably 350), PLA Mₙ 600 to 1600 (preferably) 1000;
Poly(ethylene glycol)-block-polylactide (C₁-C₄ alkyl)ether, PEG Mₙ 500 to 1100 (preferably 750), PLA Mₙ 600 to 1600 (preferably) 1000;
Poly(ethylene glycol)-block-polylactide (C₁-C₄ alkyl)ether, PEG Mₙ 600 to 900 (preferably 750), PLA Mₙ 800 to 1200 (preferably) 1000;
Poly(ethylene glycol)-block-polylactide (C₁-C₄ alkyl)ether, PEG Mₙ 700 to 900 (preferably 750), PLA Mₙ 800 to 1200 (preferably) 1000;
Poly(ethylene glycol)-block-polylactide (C₁-C₄ alkyl)ether, PEG Mₙ 750, PLA Mₙ 1000,
wherein it is preferred that the C₁-C₄ alkyl group is methyl. Hence, a preferred biocompatible amphiphilic diblock copolymer is poly(ethylene glycol)-block-polylactide methyl ether, PEG Mₙ 750, PLA Mₙ 1000.

Examples of amphiphilic block copolymers include:

poly(ethylene glycol)-block-polylactide methyl ether, PEG Mₙ 750, PLA Mₙ 1000 (also known as PEG monomethyl ether Mₙ 750 PLA Mₙ 1000);
poly(ethylene glycol)-block-polylactide methyl ether, PEG Mₙ 350, PLA Mₙ 1000;
poly(ethylene glycol)-block-polylactide methyl ether, PEG Mₙ 5000, PLA Mₙ˜5000;
poly(ethylene glycol)-block-poly(ε-caprolactone) methyl ether, PEG Mₙ 5000, poly(ε-caprolactone Mₙ 5000;
poly(ethylene glycol)-block-poly(ε-caprolactone) methyl ether, PEG Mₙ 5000, poly(ε-caprolactone Mₙ 13000;
poly(ethylene glycol)-block-poly(ε-caprolactone) methyl ether, PEG Mₙ 5000, poly(ε-caprolactone) Mₙ 32000; all of which are commercially available from Sigma-Aldrich Co.

As will be understood by the person skilled in the art, “methyl ether” refers to a methyl group at a terminal end of the PEG-chain (obviously not both ends because this would prevent the PLA from attaching to the PEG). Also the Mₙ values for the PEG, such as in “PEG mono methyl ether Mₙ 750” refer to the Mₙ of the PEG per se, so not including the additional CH₂ group of the methyl group.

Amphiphilic polymers are available form commercial sources or they may be synthesised for use in the process. The amphiphilic polymer may be a single amphiphilic polymer or a mixture comprising two or more amphiphilic polymers. The preparation of the preferred amphiphilic polymers, i.e. amphiphilic diblock copolymers having poly(alkylene glycol) (PAG) blocks (e.g. PEG blocks) can be performed in a number of ways. Known methods are disclosed in e.g. X. M. Deng et al., J. Polym. Sci, Part C, Polymer Lett. 28, 411-416, 1990; K. J. Zhu et al., J. Polym. Sci, Part C, Polymer Lett. 24, 331, 1986.

Another group of preferred stabilizing agents are gelatines. This can be any gelatine available in the market either from bone or hide from cow or pig or fish. However animal derived gelatine has some disadvantages and for pharmaceutical applications the use of recombinant produced gelatine is preferred.

As mentioned above, it is preferred that solution (I) and/or the precipitating agent (II) comprises a stabilising agent for the organic compound. Hence, either the solution (I) or the precipitating agent (II) may comprise an amphiphilic polymer, preferably an amphiphilic block copolymer, most preferably an amphiphilic diblock copolymer, or a gelatine, in particular a recombinant gelatine. According to a preferred embodiment of the present invention, the solution (I) and the precipitating agent (II) comprises an amphiphilic block copolymer and a gelatine, respectively, or vice versa.

In addition, the wetting agent, when present, is preferably selected from the group consisting of sodium dodecylsulphate, Tween 80, Cremophor A25, Cremophor EL, Pluronic F68, Pluronic L62, Pluronic F88, Span 20, Tween 20, Cetomacrogol 1000, Sodium Lauryl Sulphate, Pluronic FI27, Brij 78, Klucel, Plasdone K90, Methocel E5, PEG, Triton X100, Witconol-14F and Enthos D70-30C. In case the particles that are precipitated according to the process of the present invention have to be used in a pharmaceutical application, it is preferred that the stabilising agent and the wetting agent are biocompatible.

According to an embodiment of the present invention, the stabilising agent and/or the wetting agent may be fed to the collecting vessel instead of feeding the stabilising agent and/or the wetting agent to the closed type mixing chamber. According to another embodiment of the present invention, the stabilising agent and/or the wetting agent may be fed to both the collecting vessel and the closed type mixing chamber. According to yet another embodiment of the present invention, the wetting agent may be fed to the collecting vessel instead of to the closed type mixing chamber.

As will be apparent to the person skilled in the art, the organic compound per se need not to be used in the process according to the present invention. Obviously, it is possible to employ a precursor of the organic compound, wherein a precipitation agent is used that is capable of transforming this precursor into the organic compound per se. Consequently, according to this embodiment of the present invention, a precipitation agent is employed that is reactive with the precursor of the organic compound. This enables a substantially instantaneous chemical reaction between the precursor and the precipitating agent involving the formation of covalent or ionic bonds such as by protonation/deprotonation, by anion/cation exchange, by acid addition salt formation/liberation, redox reactions, addition reactions and the like. Obviously, by the term “substantial instantaneous” a time is intended that is substantially shorter than the average residence time of (the precursor of) the organic compound in the closed type mixing chamber.

Obviously, it is important that the solution (I) of the organic compound is very well mixed with the precipitation agent (II) so that precipitation occurs in a controlled way in the part of the mixing chamber where the oversaturation allows for precipitation. By the continuous outflow of the precipitate of the organic compound and the liquid phase, a steady state is reached within the closed type mixing chamber which can be maintained continuously. In general and preferably, the residence time in the mixing chamber is more than 0.0001 second and less than 5 seconds, preferably more than 0.001 second and less than 3 seconds. When the residence time is too long, extremely fine grains once formed in the mixing chamber grow to larger sizes and the average particle size distribution becomes undesirably wide. When the residence time is too short, too few nuclei are formed. The optimum residence time will vary from one organic compound to the other.

The solution (I) and the precipitation agent (II) can be mixed in various manners, provided that a stable mixture of the solution (I) and the precipitation agent (II) in the closed mixing chamber is obtained. In a preferred method, the solution (I) and the precipitation agent are mixed by sonication. In another preferred method, the solution (I) and the precipitation agent (II) are mixed by any stirring means, preferably by mechanical stirring means or magnetic stirring means. The mechanical stirring means is preferably rotatable within the mixing chamber and may be a rotatable blade. The rotatable blade may be in any form and may have any aspect ratio. That is that it may be in the form of a paddle where the ratio of its height and width are similar, or it may be in the form of a disc where its height is much smaller than its width. The term “width” is intended to express the diametric distance from the central axis of rotation of the mechanical stirring means, e.g. a paddle, to its outermost edge.

When mechanical or magnetic stirring means are employed, it is preferred that the volume of the stirring means is at least 10% and not more than 99%, more preferably at least 15% and not more than 95% of the volume of the closed type mixing chamber. Additionally, if mechanical mixing is employed, it is preferred that the mechanical stirring means comprises a shaft rotated by a motor on which shaft a symmetrical stirring blade is attached. A preferred size of stirrer blade is at least 50%. A more preferred size is at least 70%. An even more preferred size is between 80% and 99% and a most preferred size is between 80% and 95% of the smallest diameter of the mixing chamber.

To assist the mixing, it is preferred that the precipitate of the organic compound and the liquid phase is discharged from the mixing chamber through an outlet which is towards the opposite end of the mixing chamber from the inlets and not directly in line with the inlets. For example, the inlets may be positioned at the bottom part of the mixing chamber and the outlet may be positioned at the top part of the mixing chamber. According to an embodiment of the present invention, the inlets are below the middle line of the mixing chamber (e.g. below 30% height or below 20% height). The outlet may be above 70% height. According to another embodiment of the present invention, the outlet is or is approximately at a right angle (e.g. 80° to 100°, especially about 90°) relative to the flow of solution (I) and precipitating agent II) through the inlets. In this way, the liquids entering through the inlets do not immediately exit through the outlet without proper mixing.

The precipitate of the organic compound and the liquid phase are preferably discharged in a collecting vessel. The collecting vessel may comprise a second liquid phase comprising one or more of stabilisation agents, wetting agents, non-solvents, solvents or mixtures thereof.

In another embodiment, ripening of the precipitate of the organic compound is performed in the collecting vessel until the preferred average particle size and/or average particle size distribution is achieved. This modification or ripening can be achieved by stirring the liquid phase and the precipitate in the collecting vessel. During modification or ripening, the average particle size may increase, but the average particle size distribution usually becomes narrower which is sometimes advantageous. Modification or ripening can be controlled by various parameters, e.g. temperature, pH or ionic strength Consequently, according to this preferred embodiment, the process according to the present invention comprises a further step (e), wherein the precipitate of the organic compound and the liquid phase is discharged in a collecting vessel, wherein the precipitate of the organic compound is subjected to a ripening step.

In still another embodiment the precipitating agent comprises small particles of the compound to be precipitated. In this case larger particles can be obtained in a controlled way.

During an induction period of the precipitation process according to the present invention, the precipitating agent (II) is introduced with a continuous flow into the closed type mixing chamber and leaves the closed type mixing chamber via the single outlet to a collecting vessel. Subsequently, the solution (I) of the organic compound is introduced with a continuous flow into the closed type mixing chamber which results in an oversaturation of the organic compound thereby initiating the formation of a precipitate and a liquid phase. In the liquid phase, the oversaturation is reduced to such a level that essentially no precipitation will occur outside the closed type mixing chamber. Since the solution (I) of the organic compound and the precipitating agent (II) are fed continuously, a continuous outflow of the precipitate and the liquid phase is eventually achieved. After the induction period, a steady state is reached in the closed type mixing chamber meaning that basically the composition of the mixture within the closed type mixing chamber is stable and does essentially not change over time. Additionally, the composition of the outflow of the precipitate and the liquid phase is stable and does essentially not change over time as well.

The velocities of the inflow of solution (I) and precipitating agent (II) are not limited to high velocities. If multiple inlets are used, the velocity of one inflow may differ from the velocity of another inflow. However, in general the feed velocity of the inflow of the solution (I) and the precipitating agent (II) may be 0.01 m/s, 0.1 m/s or 1 m/s. Even velocities of 10 m/s or more than 50 m/s can be used. The advantage of this inventive method is, however, that with relatively low feed velocities small particle precipitation can be achieved. Feed velocities in case of multiple inlets need not to be equal. In contrast, in impinging jet mixers it is important and in fact essential that these feed velocities match each other. The ratio of feed velocities of solution (I) and precipitating agent (II) can be 1:99 to 99:1. During the induction period, the effluent of the closed type mixing chamber is collected until the composition of the effluent is essentially constant. As soon as a steady state is reached, the precipitate and the liquid phase are collected in a collecting vessel.

According to the invention, the organic compound to be precipitated, or precursors thereof are preferably dissolved in a solvent or solvent mixture as is disclosed above. The kind or nature of the precipitating agent (II) is dependent on the method of precipitation. In case of a solvent non-solvent precipitation, the precipitation agent is preferably a non-solvent, a mixture of non-solvents or a mixture of a non-solvent and a solvent, said mixture acting as a non-solvent. If a precipitation is caused by lowering the temperature, the precipitation agent is preferably a solvent or a solvent mixture having a temperature which initiates precipitation. In case of pH precipitation or ionic strength precipitation, the precipitation agent can be a solution having a pH or ionic strength, respectively, which initiates precipitation. In case of reaction precipitation, the precipitation agent will be a reactant which reacts with the precursor of the organic compound thereby inducing precipitation.

Söhnel and Garside (“Precipitation, Basic Principles and Industrial Applications”, Butterworth-Heinemann, 1992) have described the precipitation kinetics in a closed system using classical nucleation theory. Classical nucleation theory primarily deals with the determination of the steady state nucleation rate J, i.e. the estimation of the number of supercritical clusters formed per unit time interval in a unit volume of a thermodynamically metastable system. In general, high values of J yield a high number of particles and therefore small particle sizes.

Schmelzer and Slezov (Theoretical Determination of the Number of Clusters Formed in Nucleation-Growth Processes, Chapter 9, in “Aggregation Phenomena in Complex Systems, Ed. J. Schmelzer, G. Ropke, R. Manhke, Wiley-VCH, 1999) improved classical nucleation and growth theory by adopting less assumptions. For example, they dropped the assumption that growth of nuclei takes place one monomeric unit at a time. The oversaturation is one of the key parameters that dictate the nucleation and growth rate of solids during precipitation. Nucleation theories, however, have successfully been used for salt precipitation but they have limited success in predicting the particle size distribution of precipitated organic solids in a solvent anti-solvent precipitation. Possibly, fast secondary processes like Ostwald ripening might be responsible for this.

In most practical batch applications, a steady state can be established in a system only for a very short period of time as is discussed in the co-pending patent application PCT/GB2007/002786, filed 20 Jul. 2007.

An important factor of the process according to the present invention is the value of the oversaturation (or supersaturation). In order to make the necessary simplifications to the nucleation rate calculations, the precipitation process is treated as a plug-flow mixing process with perfect mixing at all times in the closed type mixing chamber wherein the oversaturation S₁₀ is defined above. Oversaturation in this respect is defined as the ratio between the actual concentration divided by the equilibrium concentration, meaning the concentration where the solution is just saturated. S₁₀ may be time-dependent if the flows, temperatures or concentrations are time-dependent. The 10 seconds allowed for start-up effects of unstabilised mixing chamber composition and temperature. Preferred experimental conditions are those that result in a high value for S₁₀. Depending on the compound to be precipitated, oversaturation values of more than 1.5, more than 2.5, more than 10 and even more can be advantageous. For some compounds even an oversaturation value of 100 or more can be used. The oversaturation can be controlled by parameters like temperature, concentration of the organic compound in the solution and the like as will be apparent to those skilled in the art.

The process according to the present invention is very suitable for precipitation of active pharmaceutical compounds into particles, possibly crystalline, with a small average size and a narrow particle size distribution. Small pharmaceutical particles are very suitable to be used in a medicament. Another advantage of the present invention is that the precipitated crystalline organic compounds are very pure, essentially without inclusion of impurities.

The particles obtained by the process this invention can be of an amorphous nature or can be crystalline.

The compounds which can be precipitated according to the method of this invention, are preferably pharmaceutically active compounds, preferably selected from the group consisting of anabolic steroids, analeptics, analgesics, anesthetics, antacids, anti-arrthymics, anti-asthmatics, antibiotics, anti-cariogenics, anti-cancer drugs, anticoagulants, anticofonergics, anticonvulsants, antidepressants, antidiabetics, antidiarrheals, anti-emetics, anti-epileptics, antifungals, antihelmintics, antihemorrhoidals, antihistamines, antihormones, antihypertensives, anti-hypotensives, anti-inflammatories, antimuscarinics, antimycotics, antineoplastics, anti-obesity drugs, antiplaque agents, antiprotozoals, antipsychotics, antiseptics, anti-spasmotics, anti-thrombics, antitussives, antivirals, anxiolytics, astringents, beta-adrenergic receptor blocking drugs, bile acids, breath fresheners, bronchospasmolytic drugs, bronchodilators, calcium channel blockers, cardiac glycosides, contraceptives, corticosteroids, decongestants, diagnostics, digestives, diuretics, dopaminergics, electrolytes, emetics, expectorants, haemostatic drugs, hormones, hormone replacement therapy drugs, hypnotics, hypoglycemic drugs, immunosuppressants, impotence drugs, laxatives, lipid regulators, mucolytics, muscle relaxants, non-steroidal anti-inflammatories, nutraceuticals, pain relievers, parasympathicolytics, parasympathicomimetics, prostagladins, psychostimulants, psychotropics, sedatives, sex steroids, spasmolytics, steroids, stimulants, sulfonamides, sympathicolytics, sympathicomimetics, sympathomimetics, thyreomimetics, thyreostatic drugs, vasodialators, vitamins, xanthines, and mixtures thereof. A particularly preferred organic compound is paclitaxel (also known as taxol).

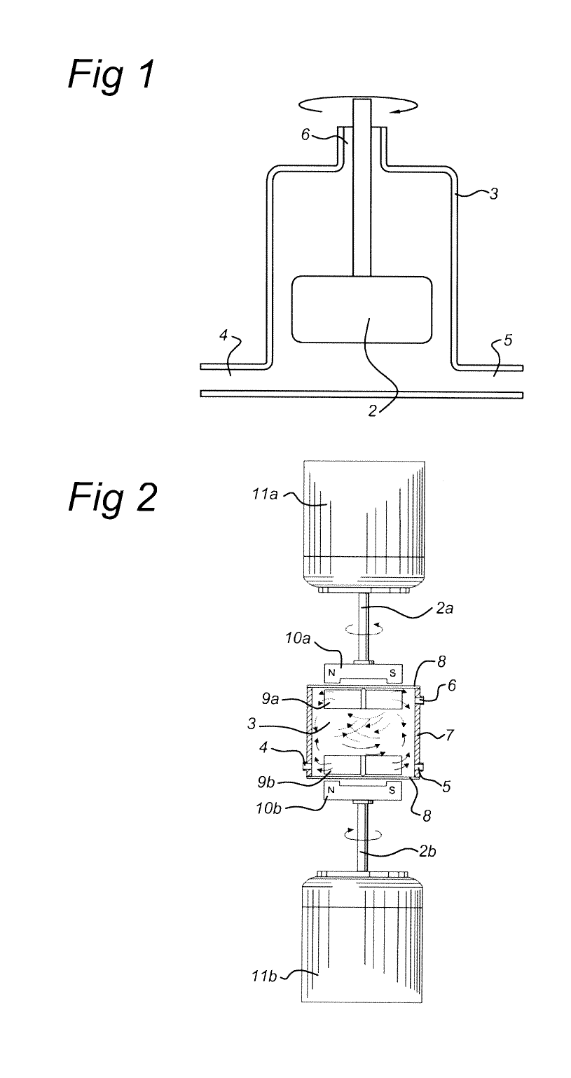

The present invention also relates to a precipitation device, preferably for performing the process according to the invention. A first embodiment of the device according to the invention is schematically disclosed in FIG. 1. The device 1 according to this first preferred embodiment comprises a stirring means 2, a closed type mixing chamber 3, a first inlet 4 for feeding a solution (I) of the organic compound in a first solvent, the inlet 4 being connected to the closed type mixing chamber 3, an second inlet 5 for feeding a precipitating agent (II) to the closed type mixing chamber 3, the inlet 5 being connected to the closed type mixing chamber 3, and a single outlet 6 for receiving a precipitate of the organic compound and a liquid phase, the outlet 6 being connected to the closed type mixing chamber 3. For illustrative purposes, the stirring means 2 is depicted as a single stirring blade, although other means that can effect stirring or mixing may be employed instead as will be appreciated by the person skilled in the art and as will be apparent below. The positions as actually depicted in FIG. 1 for inlets 4 and 5 and for outlet 6 are also shown only for illustrative purposes. However, other positions of these inlets 4 and 5 and the outlet 6 are feasible and within the scope of the present invention. In particular, the positions of the inlets 4 and 5 and of the outlet 6 determine for a part the average residence time of the organic compound in the closed mixing chamber. In general, a mixing chamber has a bottom part and a top part. Furthermore, one can define a middle line through the mixing chamber dividing the mixing chamber in a bottom part and a top part. Furthermore, one can define the lowest bottom part as 0% height, the middle line as 50% height and the very top as 100% height. Using this general description of the closed mixing chamber, the inlets 4 and 5 should be connected at the bottom part of the mixing chamber that is below the middle line for example below 30% height or 20% height. The single outlet 6 should be located at the upper part of the mixing chamber above the middle line, for example above 70% height. The inlets 4 and 5 may be diametrically opposed to each other. The inlets 4 and 5 may also be aligned in an essentially parallel fashion. The inlets 4 and 5 may also independently enter the closed type mixing chamber via the lower bottom part. Likewise, outlet 6 is depicted in FIG. 1 as being positioned at the top of the closed type mixing chamber 3, although it may also be positioned in a higher portion of a side wall of the closed type mixing chamber 3. An advantage of the embodiment where outlet 6 is positioned at the top of the closed type mixing chamber 3 is that shaft bearings are not required which may lead to contamination.

The size of the closed type mixing chamber 3 is dependent on the scale at which the precipitation is performed. On small scale one typically would use a closed mixing chamber of 0.5-150 cm³ or 0.15-100 cm³, for medium scale a closed type mixing chamber of 150-500 cm³ or 100-250 cm³ and for large scale a closed type mixing chamber of more than 500 cm³ to 1000 cm³ or even 1 m³ can be used. Preferably, the size of the closed type mixing chamber is 1 cm³-1 dm³.

Preferably, at least one stirring means is positioned between the inlets such that it acts as a physical barrier between the incoming flows of the solution (I) and the precipitating agent (II). In this way, the stirring means reduces the chance of precipitate formation at the inlets which could otherwise block these inlets. Instead, the flows of the solution (I) and the precipitating agent (II) come into contact in a circumferential instead of a “head-on” manner.

The device 1 is preferably provided with or may be connected to a collecting vessel. The collecting vessel preferably comprises a stirring means. Optionally, the closed type mixing chamber may be surrounded by the collecting vessel. Alternatively, the closed type mixing chamber may be positioned adjacent to or remote from the collecting vessel, dependent from the preference of the user. As will be obvious to the person skilled in the art, the device 1 of the present invention and/or the collecting vessel can be provided by control means to control temperature in e.g. the closed type mixing chamber and the collecting vessel, respectively. Such control means can for example be used to control the temperature of the solution (I), the precipitating agent (II), the closed type mixing chamber 3 and the supply tanks.

The device 1 of this invention comprises a supply tank (not shown) comprising the solution (I) of the organic compound and a supply tank (not shown) comprising the precipitation agent (II). The supply tanks are connected to the closed type mixing chamber by feed lines which can be hoses or fixed pipes. The transportation to the mixing chamber is done with a continuous flow provided by a pump. The pump can be any pump known in the art as long as the pump can provide a stable flow during a prolonged period of time. Suitable pumps are for example plunger pumps, peristaltic pumps and the like.

The shape of the closed type mixing chamber can in principle be chosen freely and in case it is rotationally symmetric around a central axis, it can for example be specified by two identical surfaces. i.e. one top surface and one bottom surface, at a distance x from each other which surfaces may have any shape from rectangular to dodecagonal or circular with, when applicable, a minimum diameter of Dₘᵢₙ. For example, for a closed type mixing chamber having a square shape, Dₘᵢₙ is the distance between opposite sides. In this embodiment, x can be larger than Dₘᵢₙ, and alternatively, x can also be smaller than Dₘᵢₙ. In a further embodiment, the top surface and bottom surface need not to be identical, but one surface can be for example of a smaller size than the other.

A preferred embodiment of the device according to the present invention is shown in FIG. 2. This more preferred embodiment is essentially the apparatus disclosed in U.S. Pat. No. 5,985,535, expressly incorporated by reference herein. In FIG. 2, the device 1 comprises stirring means 2a, 2b, a closed type mixing chamber 3 consisting of a tank body 7 having a central axis of rotation facing in top and bottom directions and seal plates 8 which function as tank walls sealing top and bottom opening ends of the tank body 7. The tank body 7 and the seal plates 8 are preferably made of nonmagnetic materials which are excellent in magnetic permeability if magnetic stirring is employed which will be elucidated in more detail below. Stirring means 2a, 2b are provided with outer magnets 10a, 10b and are disposed outside at the top and bottom ends of the closed type mixing chamber 3 which are essentially opposite to each other. The outer magnets 10a, 10b are coupled to stirring blades 9a, 9b inside the closed mixing chamber via magnetic forces. Motors 11a and 11b drive the outer magnets 10a and 10b in converse directions. By this, stirring blades 9a, 9b rotate in converse directions in the closed mixing chamber.

Further, in FIG. 2, the closed type mixing chamber 3 is provided with a first inlet 4 for feeding a solution (I) of the organic compound in a first solvent, the inlet 4 being connected to the closed type mixing chamber 3, an second inlet 5 for feeding a precipitating agent (II) to the closed type mixing chamber 3, the inlet 5 being connected to the closed type mixing chamber 3, and a single outlet 6 for receiving a precipitate of the organic compound and a liquid phase, the outlet 6 being connected to the closed type mixing chamber 3. Although inlets 4 and 5 are shown in a diametrically opposed fashion, they may also be aligned in an essentially parallel fashion. As the shape of the closed type mixing chamber 3, a cylindrical shape is often used, but rectangular, hexagonal and various other shapes may obviously be used as will be apparent to those skilled in the art. Likewise, the stirring means 2a, 2b driving the stirring blades 9a, 9b are shown as being disposed at the opposite top and bottom ends of the closed type mixing chamber 3, but they may obviously be disposed at the opposite left and right sides, or may be disposed diagonally, depending on the shape of the mixing chamber. Additionally, the closed type mixing chamber 3 may comprise more pairs of conversely rotating stirring blades.

In another embodiment of the device according to FIG. 2, an odd number of magnetic stirring devices may be used, e.g. one, three or five magnetic stirring means. Furthermore, the use of pairwise oriented stirring means in combination with a single stirring means may lead to even more efficient stirring.

According to the invention, a preferred process for the precipitation of an organic compound that is employed in a device according to FIG. 2 comprises the following steps:
(I) feeding a flow (i) comprising a solution (I) comprising the organic compound and a first solvent via a first inlet to a closed type mixing chamber and contacting flow (i) with a flow (ii) comprising a precipitating agent (II) fed simultaneously with flow (i) via a second inlet to the closed type mixing chamber thereby forming a flow (iii) comprising a precipitate of the organic compound and a liquid phase; and
(II) discharging flow (iii) comprising the precipitate of the organic compound and the liquid phase from the closed type mixing chamber, preferably in a geometric direction cocurrent with the direction by which flow (i) comprising the solution of the organic compound is fed to the closed type mixing chamber, via a single outlet.

The term “cocurrent direction” is to be understood that the direction of flow (iii) is not countercurrent to the direction of flow (i). The term “cocurrent direction” is more in particular to be understood as that the angle defined by the axis of flow (i) and the axis of flow (iii) varies from 90° to 180°.

In this preferred process, it is further preferred that flow (ii) comprising the precipitating agent (II) is fed to the closed type mixing chamber in a direction essentially diametrically opposed to the direction by which the flow (i) comprising the solution (I) comprising the organic compound is fed to closed type mixing chamber.

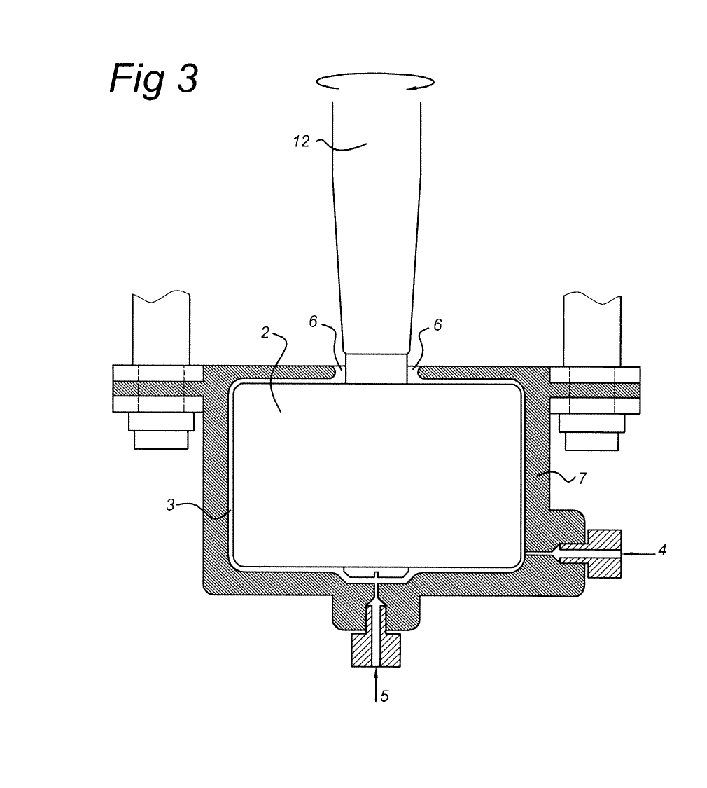

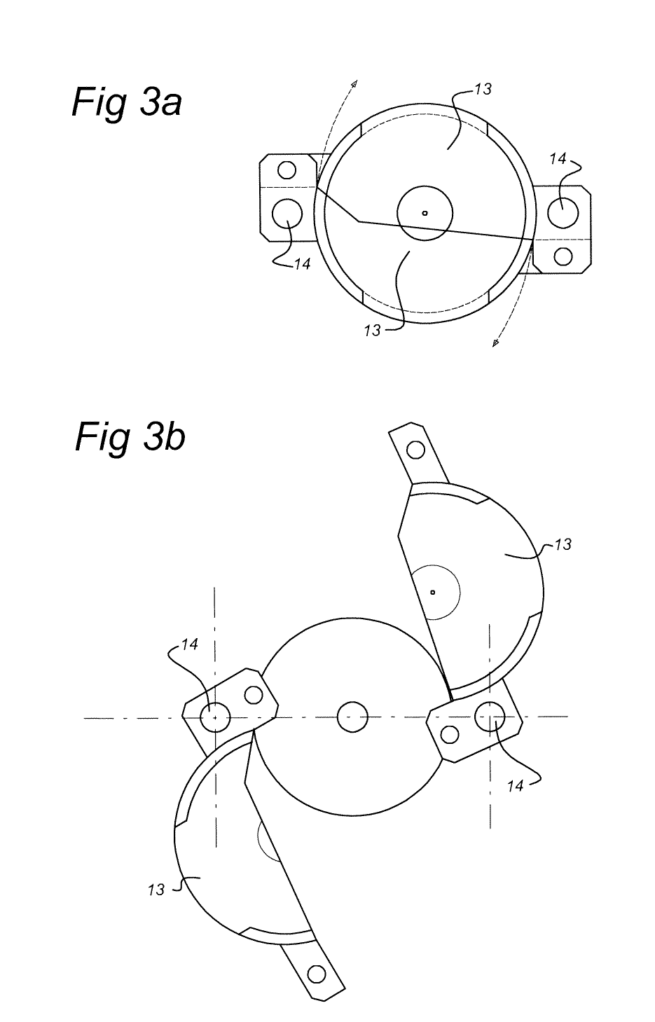

Another preferred embodiment of the device according to the present invention is disclosed in FIG. 3. In FIG. 3, the device 1 comprises a stirring means 2, a closed type mixing chamber 3 consisting of a tank body 7 having a central axis of rotation facing in top and bottom directions. Stirring means 2 is disposed preferably in the centre of the closed type mixing chamber 3 and can be driven preferably directly via a stirrer axis 12 and a motor (not shown). The inlets 4 and 5 are preferably essentially perpendicular to each other. However, the positions of inlets 4 and 5 are interchangeable, that is that inlet 4 may enter the closed type mixing chamber 3 via the bottom thereof whereas inlet 5 may enter the closed type mixing chamber 3 via a sidewall. Alternatively, inlet 5 may enter the closed type mixing chamber 3 via the bottom thereof whereas inlet 4 enters the closed type mixing chamber 3 via a sidewall. It is also possible that both inlets 4 and 5 enter through the side wall, in which the angle in a horizontal plane between the inlets can have any value, but is preferably between 90° and 180° In this embodiment the stirrer axis or shaft 12 is positioned within the single outlet 6 of the closed type mixing chamber 3. It is further possible that both inlets 4 and 5 enter via the bottom part of the closed type mixing chamber 3. In a preferred embodiment, inlet 5 via which the anti solvent enters the closed type mixing chamber 3 is placed at the bottom so that undesired precipitation at the inlet into the reaction chamber is prevented.

Additionally, in this embodiment of the device it is also highly preferred that the volume of the stirring means 2 is at least 10% and not more than 99%, preferably not more than 95%, of the volume of the closed type mixing chamber 3. Hence, this preferred embodiment of the precipitation device 1 of the present invention comprises a stirring means 2 comprising an axis or shaft 12, a closed type mixing chamber 3 consisting of a tank body 7 having a central axis of rotation facing in top and bottom directions, an inlet 4 and an inlet 5 that are preferably essentially perpendicular to each other, and an outlet 6 in which axis or shaft 12 of stirring means 2 is positioned.

The device according to the embodiment of FIG. 3 may be constructed from moveable parts as is shown in FIGS. 3A and 3B showing a top view of this embodiment of device 1. Here, the closed type mixing chamber 3 is formed by two moveable chamber parts 13 that are rotatable around hinges 14. The movable chamber parts 13 interlock around the stirring means 2.

According to the invention, a preferred process for the precipitation of an organic compound that is employed in a device according to FIG. 3 comprises the following steps:
(I) feeding a flow (i) comprising a solution (I) comprising the organic compound and a first solvent via a first inlet to a closed type mixing chamber and contacting flow (i) with a flow (ii) comprising a precipitating agent (II) fed simultaneously with flow (i) via a second inlet to the closed type mixing chamber thereby forming a flow (iii) comprising a precipitate of the organic compound and a liquid phase; and
(II) discharging flow (iii) comprising the precipitate of the organic compound and the liquid phase from the closed type mixing chamber in a geometric direction essentially perpendicular to either the direction by which flow (i) comprising the solution of the organic compound is fed to the closed type mixing chamber or the direction by which flow (ii) comprising the precipitating agent (II) is fed to the closed type mixing chamber.

Alternatively, step (II) may also comprise discharging flow (iii) comprising the precipitate of the organic compound and the liquid phase from the closed type mixing chamber in a geometric direction essentially cocurrent with either the direction by which flow (i) comprising the solution of the organic compound is fed to the closed type mixing chamber or the direction by which flow (ii) comprising the precipitating agent (II) is fed to the closed type mixing chamber or with both if both inlets enter the closed type mixing chamber via its bottom part.

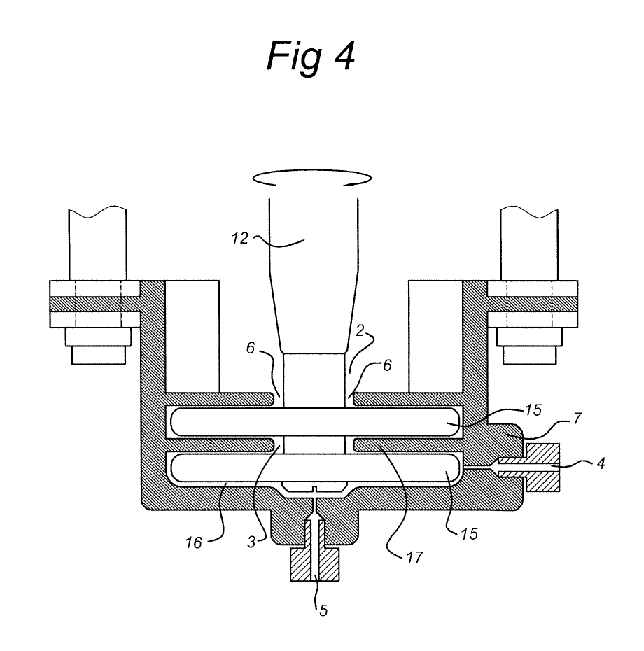

Another preferred embodiment of the device according to the present invention is disclosed in FIG. 4. Also this embodiment may be constructed from moveable parts as is shown in FIGS. 3A and 3B as will be apparent to the person skilled in the art.

In FIG. 4, the device 1 comprises a stirring means 2, a closed type mixing chamber 3 consisting of a tank body 7 having a central axis of rotation facing in top and bottom directions. Also in this embodiment the stirrer axis or shaft 12 is positioned within the single outlet 6 of the closed type mixing chamber 3. The inlets 4 and 5 are preferably essentially perpendicular to each other. However, also in this embodiment the positions of inlets 4 and 5 are interchangeable and also in this embodiment inlets 4 and 5 may enter the closed type mixing chamber through the side walls or via the bottom part of the closed type mixing chamber. Here it is preferred that the precipitating agent (II) enters via the bottom part of the mixing chamber.

Additionally, in this embodiment of the device, it is also highly preferred that the volume of the stirring means 9 is at least 10% and not more than 99%, preferably not more than 95%, of the volume of the closed type mixing chamber 3. In the embodiment shown stirring means 2 comprises two stirring disks 15 and the closed type mixing chamber 3 comprises a mixing zone which is divided in compartments by separating wall 17. A mixing chamber with one disk can also be used, while also mixing chambers having three or more compartments, each compartment being provided with a stirring disk attached to one single axis, can be used. Hence, this preferred embodiment of the device 1 of the present invention comprises a stirring means 2 comprising an axis or shaft 12 and at least one, two, three, four or more stirring disks 15, a closed type mixing chamber 3 consisting of a tank body 7 having a central axis of rotation facing in top and bottom directions, said closed type mixing chamber 3 comprising a mixing zone 16, an inlet 4 and an inlet 5 that are preferably essentially perpendicular to each other, and an outlet 6 in which axis or shaft 12 of stirring means 2. Optionally, the mixing zone 16 may be divided in compartments by one or more separating walls 17. Within the scope of this embodiment are devices comprising more than one stirring disk with only a single mixing zone, i.e. a mixing zone that is not separated into one or more compartments by one or more separating walls, as well as devices comprising more than one stirring disk and a mixing zone separated into several compartments by one or more separating walls. Obviously, if the device comprises only a single stirring disk, it will generally not comprise a separating wall, so that the mixing zone comprises only one compartment.

Also the embodiment according to FIG. 4 allows for a process comprising the following steps:
(I) feeding a flow (i) comprising a solution (I) comprising the organic compound and a first solvent via a first inlet to a closed type mixing chamber and contacting flow (i) with a flow (ii) comprising a precipitating agent (II) fed simultaneously with flow (i) via a second inlet to the closed type mixing chamber thereby forming a flow (iii) comprising a precipitate of the organic compound and a liquid phase; and
(II) discharging flow (iii) comprising the precipitate of the organic compound and the liquid phase from the closed type mixing chamber in a geometric direction essentially perpendicular to either the direction by which flow (i) comprising the solution of the organic compound is fed to the closed type mixing chamber or the direction by which flow (ii) comprising the precipitating agent (II) is fed to the closed type mixing chamber.

Like the embodiment of the FIG. 3, step (II) may also comprise discharging flow (iii) comprising the precipitate of the organic compound and the liquid phase from the closed type mixing chamber in a geometric direction essentially cocurrent with either the direction by which flow (i) comprising the solution of the organic compound is fed to the closed type mixing chamber or the direction by which flow (ii) comprising the precipitating agent (II) is fed to the closed type mixing chamber or with both if both inlets enter the closed type mixing chamber via its bottom part.

Preferably, all parts of the closed type mixing chamber that are in contact with the mixture in the closed type mixing chamber are coated with a layer of a material that prevents adhering, fouling, incrustation and such. Preferred materials are those having moisture absorption according to ASTM D 570 at a relative humidity of 50% and a temperature of 23° C. of less than 1%. Suitable examples of such materials include fluorinated alkene polymers and copolymers, e.g. polytetrafluoroethylene, and polyacetals, e.g. polyoxymethylene.

At the start of the nucleation, nuclei are surrounded by over-saturated fluid. When two or more of these particles stay in contact for too long, they will be “cemented” together to an agglomerate. Furthermore, unlike inorganic particles in aqueous media, organic particles are usually not electrically charged and therefore these organic particles do not have a repulsive mechanism. In the present invention, the drag/shear forces in the closed type mixing chamber imposed on the nuclei by the fluid motion prevents the particles from agglomerating. In one embodiment of this invention, excessive turbulence is used to reduce the inter-particle contact times to values that do not allow agglomeration while the surrounding fluid is still over-saturated.

According to the present invention, it is preferred that the diameter of the stirring means is at least 50% and more preferably at least 70% and most preferably at least between 80% and 99% of the smallest diameter of the closed type mixing chamber. Very good results were obtained with a stirring means which had a diameter of about 90% to 95% of the smallest diameter of the mixing chamber. In another embodiment, very good results were obtained with a stirring means which had a diameter of 80% to 90% of the smallest diameter of the closed type mixing chamber. The stirring means is here preferably a mechanical stirring means.

In case a type of mixer is used with a perforated or closed blade, with or without added components on the blade surface, having a symmetry around the axis, around which the rotations happens, the mixing can be characterized by the impeller Reynolds number N_{Re}, which is given by the equation:

$${N}_{Re} = \frac{{Da}^{2} \cdot N \cdot ρ}{μ}$$

wherein:

Da=the blade diameter;

N=rotational speed;

ρ=fluid density; and

μ=viscosity.

Typically in this case the flow is isotropic turbulent when N_{Re} is larger than 10⁴, see Perry (Perry's Chemical Engineers' Handbook, Ed.: R. H. Perry and D. W. Green, McGraw-Hill, Ch18, 1999).

From the formula above it appears that the Reynolds number increases at higher stirrer blade diameter. In the present invention it was found, that a preferred diameter of stirrer blade is at least 50% and more preferably at least 70% and most preferably between 80 and 99% of the smallest diameter of the mixing chamber. Very good results were obtained with a stirrer blade which had a diameter of around 90% to 95% of the smallest diameter of the mixing chamber.

In the present invention, when opposite stirring means are driven in the closed type mixing chamber as is shown in the embodiment of the device according to FIG. 2, it is necessary to rotate stirring blades at high speed for obtaining a high mixing efficiency. The rotation speed is 1,000 rpm or more, preferably 3,000 or more, and more preferably 5,000 rpm or more. A pair of conversely rotating stirring means may be rotated at the same rotating speed or at different rotating speeds. In case of a stirrer means which is symmetrical around an axis, the stirrer speed should be more than 500 rpm, for example 1,000 rpm or 5,000 or even 10,000 rpm. Nowadays, stirrers are commercially available having a stirrer speed of 20,000 rpm and even more. In general, the higher the stirring speed the better the mixing and therefore it is not possible to define an upper limit for the preferred stirring speed.

The residence time of the organic compound in the closed type mixing chamber can be varied amongst others by changing various parameters, e.g. the inflow of the solution (I) of the organic compound, the inflow of the precipitation agent (II), the choice of the type, e.g. shape and size, of the stirring means, intensity of mixing and positions of the inlets and the single outlet. A too short residence time in the closed type mixing chamber is undesired as it will result in uncontrolled nucleation outside the closed mixing chamber due to the fact that an over-saturated mixture will enter the collecting vessel. A too long residence time in the closed type mixing chamber is also undesired as it will result in excessive agglomeration and growth. Solvent and non-solvent, together with for example temperature, can be selected to control the rate of the nucleation. The induction time can for example be from 10⁻⁹ to 10⁻² seconds. The mixing is therefore a very important factor, as with reduced mixing efficiencies at these very high nucleation rates, agglomeration is almost inevitable.

Also for compounds not having such a fast nucleation time, the residence times in the closed type mixing chamber should not be too long, because the efficiency of the precipitation process will be low. Furthermore, a long residence time results in a wide average particle size distribution and larger particles. In practice, the mixing chamber residence time preferably does not exceed 3 seconds and is below 1 second. In case nucleation proceeds slowly, e.g. from 10⁻³ until 10⁻⁶ seconds, the conditions are preferably chosen such that the residence time is more than 0.1 but below 5 seconds, more preferably below 3 seconds and even more preferably below 1 second.

The residence time t is calculated as follows:

t=v/(a+b)

wherein:
v: The volume of the mixing space of a mixing vessel (cm³)
a: The addition flow of an organic compound solvent solution (cm³/sec)
b: The addition flow of the precipitation-agent(cm³/sec)

Preferably, the precipitated organic compound arising from the process according to the present invention has an average particle size of less than 1 μm, more preferably less than 700 nm, especially less than 500 nm, more especially less than 200 nm. Preferably, the precipitated organic compound has a unimodal particle size distribution.

The process according to the present invention may also include a step of drying the precipitated organic compound, e.g. by using a spray drier. The drying of the precipitated organic compound can start within 10 minutes of performing step (c), or within 5 minutes, or within 2 minutes and also within 1 minute of performing step (c). The drying can also be postponed as the precipitates are very stable in the presence of the amphiphilic block copolymers. The process according to the present invention may be performed on any scale and steps (a) to (d) may be performed continuously. In this way, large quantities of the desired particulate organic compound may be prepared, including on an industrial scale. There is no need to include jets in the process which have to be aligned carefully.

Preferably, the precipitated organic compound is in particulate form and has a D50 of less than 500 nm, more preferably less than 400 nm, even more preferably of less than 300 nm, and in particular less than 200 nm. The D50 can be measured by techniques known in the art, e.g. laser diffraction according to method ISO 13320-1 using e.g. a Malvern Mastersizer 2000 particle size analyser.

The present invention also provides a process for the manufacture of a medicament comprising performing the process according to the invention wherein the organic compound is a pharmaceutically active organic compound. The process for the manufacture of the medicament preferably includes mixing the pharmaceutically active organic compound with a pharmaceutically acceptable carrier or excipient.

The process according to the present invention may also include a step of sterilising the pharmaceutically active organic compound, preferably after it has been dried. Typical sterilisation techniques include irradiation, heating and filtration.

The invention also encompasses a method of treating a mammal in need thereof comprising administering of a medicament obtainable by the process according to the present invention. This medicament is preferably for the treatment of cancer.

### EXAMPLES

All following chemicals were obtained from Sima-Aldrich Co., Zwijndrecht, The Netherlands:

Paclitaxel from taxus brevifolia, ≧95% (HPLC),

### Pregnenolone, ≧98%,

Fenofibrate, ≧95%, powder,

### Cyclosporin A, BioChemika, ≧98.5% (TLC),

Tetrahydrofuran (THF), biotech grade, ≧99.9%, inhibitor-free,
Citric acid, USP grade,
D-Mannitol, USP grade,
The MPEG-PLA block copolymers,
Anhydrous ethanol (100% DAB), PH.EUR, was obtained from Boom B.V., Meppel, The Netherlands,
Fish gelating 150 kDa was obtained from Norland Products Inc., Cranbury, USA, Hydrolysed fish gelatine 4.2 kDa was obtained from Nitta Gelation Inc., Japan, The water used was purified by demineralisation and filtration techniques onsite.

### Comparative Example 1

The organic compound is pregnenolone, precipitated from ethanol/water in a 3.75 litre CSTR.

Tank anti-solvent solution initial volume V₀=1500 cm³ pure water with a temperature of T=275 K, initial volume fraction of the solvent ethanol x₀=0 (0% EtOH in water, T=275 K), pregnenolone solution feed rate is 1000 cm³/min, pregnenolone concentration 34 g/l in EtOH (=saturation conc. at 325 K), feed temperature T=328 K, stirrer rotational speed 750 rpm. Total batch addition time is 45 seconds. S₁₀=190.

Turbidity is observed immediately after addition start.

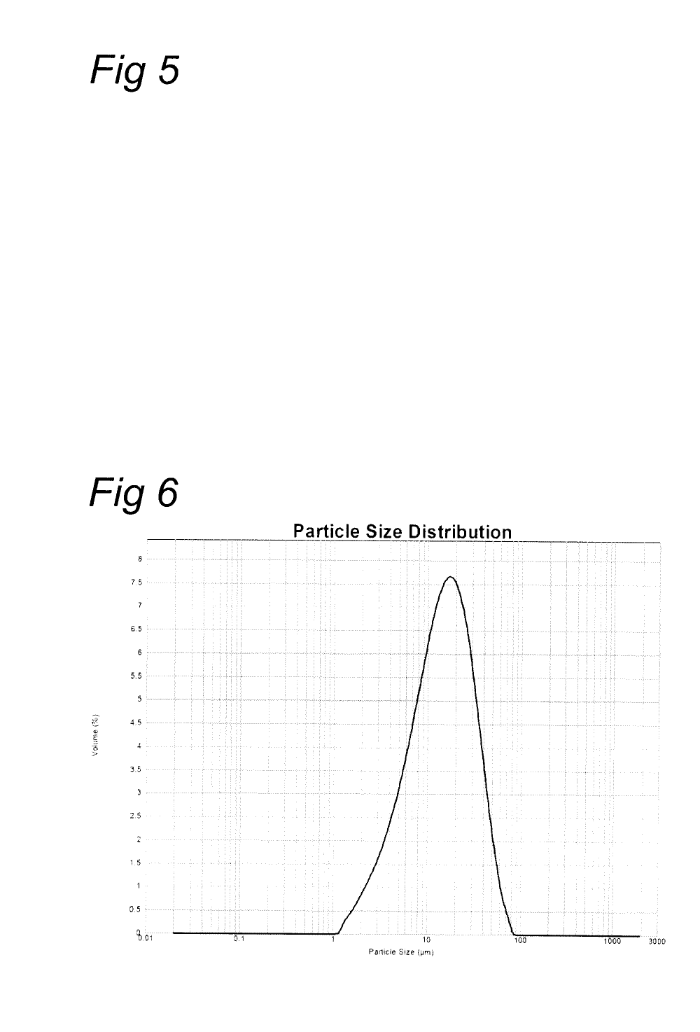

The particle size distribution is wide, including many particles of 10 μm edge length or more. FIG. 5 shows the crystals that were obtained. FIG. 6 shows the average particle size distribution that was measured with a Malvern Mastersizer 2000. The D50 of this batch is 14.59 μm. So 50% of all particles have a volume median size which is smaller than 14.59 μm. The D90 of this batch is 36.22 μm.

### Inventive Example 1

The organic compound is pregnenolone, precipitated from ethanol/water in a mixing apparatus according to FIG. 1 with a stirring tank internal volume of 0.7 cm³.

The pregnenolone solution feed rate is 10 cm³/min, pregnenolone concentration 34 g/l in EtOH (=saturation conc. at 325 K), feed temperature T=328 K, both stirrers are operated at 6,000 rpm in opposite direction. The anti-solvent water with a temperature of T=275 K is fed with a flow of 110 cm³/min. The total batch addition time to make 100 cm³ is 50 seconds. S₁₀=200.

Turbidity is observed immediately after addition start.

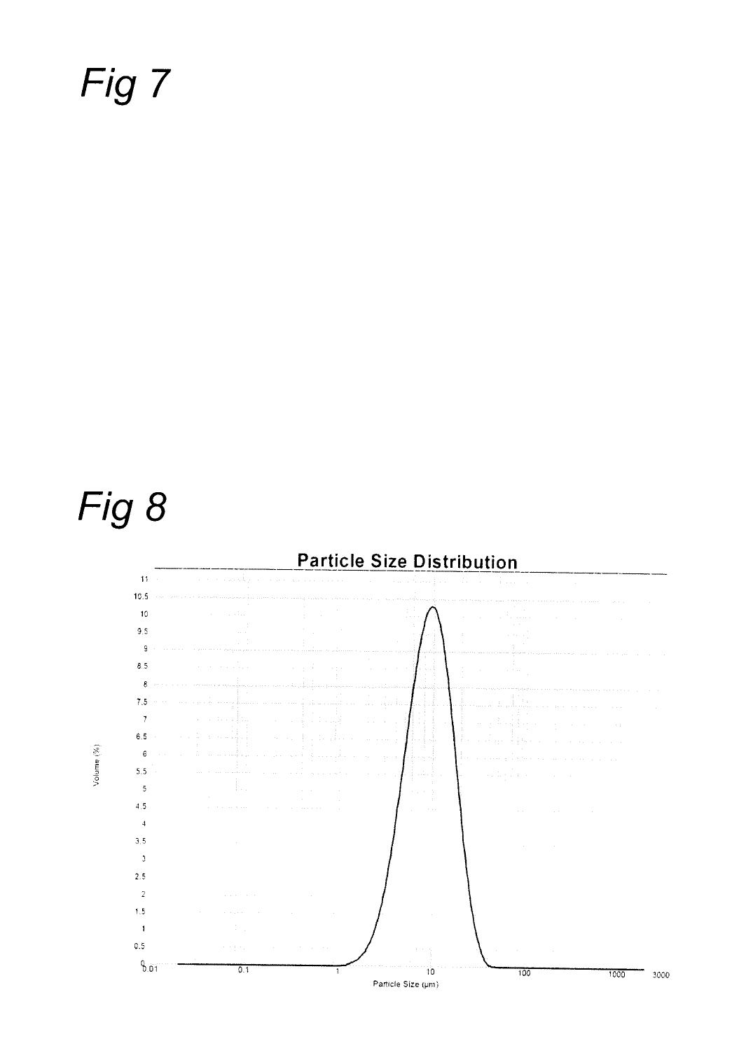

The particle size distribution is narrow and the average particle size is much lower than if precipitated in the CSTR. FIG. 7 shows the crystals that were obtained.

Particle size distribution was measured with a Malvern Mastersizer 2000. The D50 of this batch is 9.17 μm. The D90 of this batch is 18.72 μm. (FIG. 8)

### Inventive Example 2

The organic compound is pregnenolone, precipitated from ethanol/water in a mixing apparatus according to FIG. 2 with a stirring tank internal volume of 0.7 cm³. The pregnenolone solution feed rate is 10 cm³/min, pregnenolone concentration 34 g/l in EtOH (=saturation conc. at 325 K), feed temperature T=328 K, both stirrers are operated at 6,000 rpm in opposite direction. The anti-solvent water contains 4% of a hydrolysed non-gelling fish gelatine, molecular weight average 20 kDa. The gelatine solution at a temperature of T=275 K is fed with a flow of 110 cm³/min. The total batch addition time to make 100 cm³ is 50 seconds. S₁₀=200.

Turbidity is observed immediately after addition start.

The particle size distribution is bimodal and the average particle size is much lower than if precipitated without the stabilizing gelatine.

Particle size distribution was measured with a Malvern Mastersizer 2000. The D50 of this batch is 1.36 μm. The D90 of this batch is 4.58 μm.

### Inventive Example 3

The organic compound is fenofibrate, precipitated from ethanol/water in a mixing apparatus according to FIG. 2 with a stirring tank internal volume of 0.7 cm³.

The fenofibrate solution feed rate is 10 cm³/min, fenofibrate concentration 20 g/l in EtOH (saturation conc. at 293K is 40 g/l), feed temperature T=293 K, both stirrers are operated at 6,000 rpm in opposite direction. The anti-solvent water contains 4% of a non-hydrolysed non-gelling fish gelatine, molecular weight average 150 kDa. The gelatine solution at a temperature of T=293 K is fed with a flow of 110 cm³/min. The total batch addition time to make 100 cm³ is 50 seconds. S₁₀=4355.

Turbidity is observed immediately after addition start.

The particle size distribution is unimodal and the average particle size is in the nanometer range. Particle size distribution was measured with a Malvern Mastersizer 2000. The D50 of this batch is 127 nm. The D90 of this batch is 228 nm.

### Inventive Example 4

The organic compound is fenofibrate, precipitated from ethanol/water in a mixing apparatus according to FIG. 2 with a stirring tank internal volume of 0.7 cm³.

An ethanolic solution containing 20 g/l fenofibrate and 4.4 g/l poly(ethylene glycol)-block-polylactide methyl ether (PEG Mₙ 750, PLA Mₙ 1000; commercially available from Aldrich) is prepared and set at a temperature of 293 K. The anti-solvent water contains 4% of a non-hydrolysed non-gelling fish gelatin, molecular weight average 150 kDa. This anti-solvent solution is set at a temperature of T=293 K.

The fenofibrate/block-copolymer solution feed rate is 10 cm³/min. The gelatin solution is fed with a flow of 110 cm³/min. The stirrer blades had diameters of 83% of the chamber diameter. Both stirrers are operated at 6,000 RPM in opposite direction. The total batch addition time to make 100 cm³ is 50 seconds. S₁₀=4355.

Turbidity is observed immediately after addition start.

The particle size distribution (measured using a Malvern Mastersizer 2000) is unimodal and the average particle size is in the nanometer range. Particle size distribution was measured with a Malvern Mastersizer 2000. The D50 of this batch is 111 nanometer. The D90 of this batch is 206 nanometer.

### Inventive Example 5

A solution was prepared comprising THF and paclitaxel (10 g/l) and poly(ethylene glycol)-block-polylactide methyl ether (PEG Mₙ 5000, PLA Mₙ 5000) (10 g/l) at 20° C. The precipitating agent was pure water at 0° C.

The precipitation was performed as in Inventive Example 4, wherein the solution feed rate was 15 cm³/min and the precipitating agent feed rate was 105 cm³/min. The ratio of solvent solution to precipitating agent was 20:100.

The initial particle size (D50) of the resultant particles was about 260 nm.

### Inventive Example 6

A solution was prepared comprising THF and paclitaxel (10 g/l) and poly(ethylene glycol)-block-polylactide methyl ether (PEG Mₙ 350, PLA Mₙ 1000) (10 g/l) at 20° C. The precipitating agent was pure water at 0° C.

The precipitation was performed as in Inventive Example 4, wherein the solution feed rate was 15 cm³/min and the precipitating agent feed rate was 105 cm³/min. The ratio of solvent solution to precipitating agent was 15:105.

The initial particle size (D50) of the resultant particles was about 123 nm.

### Inventive Example 7

A solution was prepared comprising THF and paclitaxel (10 g/l) and poly(ethylene glycol)-block-polylactide methyl ether (PEG Mₙ 750, PLA Mₙ 1000) (10 g/l) at 20° C. The precipitating agent was pure water at 0° C.

The precipitation was performed as in Inventive Example 4, wherein the solution feed rate was 15 cm³/min and the precipitating agent feed rate was 105 cm³/min. The ratio of solvent solution to precipitating agent was 15:105.

The initial particle size (D50) of the resultant particles was below 115 nm.

### Inventive Example 8

A solution was prepared comprising THF and cyclosporine A (10 g/l) and poly(ethylene glycol)-block-polylactide methyl ether (PEG Mₙ 750, PLA Mₙ 1000) (10 g/l) at 20° C. The precipitating agent was a 1 wt % solution of citric acid in pure water at 0° C.

The precipitation was performed as in Inventive Example 4, wherein the solution feed rate was 15 cm³/min and the precipitating agent feed rate was 105 cm³/min. The ratio of solvent solution to precipitating agent was 15:105.

The initial particle size (D50) of the resultant particles was about 132 nm.

### Comparative Example 2

The method of Inventive Example 4 was repeated using an amphiphilic polymer that was not a block copolymer, except that solution (I) was a solution of pregnenolone in EtOH (34 g/l) at 50° C. and the precipitating agent was water containing 4 wt. % of ydrolysed non-gelling fish gelatine (4.2 kDa). The total batch addition time to make 100 cm³ of product was 50 s.

The particle size distribution was measured with a Malvern Mastersizer 2000. The particles bad a bimodal particle size distribution. The D50 of the particles was 1.36 μm. The D90 of the particles was 4.58 μm.

### Inventive Example 9

A solution was prepared comprising THF and paclitaxel (10 g/l) and poly(ethylene glycol)-block-polylactide methyl ether (PEG Mₙ 750, PLA Mₙ 1000) (10 g/l) at 20° C. The precipitating agent was water containing citric acid (1 wt. %) and D-mannitol (5 wt. %) at 0° C.

The precipitation was performed as in Inventive Example 4, wherein the solution feed rate was 15 cm³/min and the precipitating agent feed rate was 105 cm³/min. The ratio of solvent solution to precipitating agent was 15:105.

The initial particle size (D50) of the resultant particles was 118 nm.

### Comparative Example 3

The method of Inventive Example 4 was repeated without using an amphiphilic polymer, except that solution (I) was a solution of pregnenolone in EtOH (34 g/l) at 50° C. and that the precipitating agent was water. The total batch addition time to make 100 cm³ of product was 50 s.

The particle size distribution was measured with a Malvern Mastersizer 2000. The particles had a narrower particle size distribution than Comparative Example 2 and the average particle size was in the nanometer range. The D50 of the particles was 9.17 μm. The D90 of the particles was 18.72 μm.

1-34. (canceled)

35. A process for the precipitation of an organic compound, the process comprising:
(a) introducing a solution of the organic compound in a first solvent via a first inlet into a closed type mixing chamber;
(b) introducing, simultaneously with step (a), a precipitation agent via a second inlet into the closed type of mixing chamber;
(c) mixing the solution and the precipitating agent by sonication, by a rotatable magnetic stirring means or by a rotatable mechanical stirring means thereby forming a precipitate of the organic compound and a liquid phase; and
(d) discharging the precipitate and the liquid phase from the closed type mixing chamber via a single outlet.

36. The process according to claim 35, wherein the solution and/or the precipitating agent comprises a stabilising agent selected from the group consisting of a gelatine, a recombinant gelatine, an amphiphilic polymer, an oligonucleotide, a RNA sequence, a DNA sequence, a polysaccharide, an oligosaccharide, a disaccharide, a monosaccharide, a fatty acid, and mixtures thereof, wherein said amphiphilic polymer is an amphiphilic block copolymer comprising hydrophilic and hydrophobic blocks, said hydrophilic blocks being polyethylene glycol (PEG) and/or polyethylene glycol (PEG) ether blocks and said hydrophobic blocks being polylactide (PLA) blocks, wherein said PLA block has a Mₙ of 250 to 5000.

37. The process according to claim 35, wherein the organic compound is a pharmaceutically active organic compound.

38. The process according to claim 36, wherein the stabilising agent is a recombinant produced gelatine.

39. The process according to claim 36, wherein the stabilising agent is an amphiphilic block copolymer comprising hydrophilic and hydrophobic blocks, said hydrophilic blocks being polyethylene glycol (PEG) and/or polyethylene glycol (PEG) ether blocks and said hydrophobic blocks being polylactide (PLA) blocks, wherein said PLA block has a Mₙ of 250 to 5000.

40. The process according to claim 35, wherein the solution comprises a mixture of solvents.

41. The process according to claim 35, wherein the precipitating agent comprises a non-solvent, a mixture of non-solvents or a mixture of a non-solvent and a solvent.

42. The process according to claim 35, wherein the solution and/or the precipitation agent comprises a wetting agent.

43. The process according to claim 42, wherein the wetting agent is a biocompatible wetting agent.

44. The process according to claim 35, wherein the solution comprises a precursor of the organic compound.

45. The process according to claim 44, wherein the precipitation agent comprises a compound that is reactive with the precursor of the organic compound.

46. The process according to claim 35, wherein the organic compound is retained in the mixing chamber between 0.0001 and 5 seconds.

47. A device for the precipitation of an organic compound comprising:
(a) a closed type mixing chamber;
(b) a first inlet for feeding a solution of the organic compound in a solvent, the inlet being connected to the closed type mixing chamber;
(c) a second inlet for feeding a precipitating agent to the closed type mixing chamber, the inlet being connected to the closed type mixing chamber;
(d) a single outlet for receiving a precipitate of the organic compound and a liquid phase, the outlet being connected to the closed type mixing chamber; and
(e) a mechanical stirring means, wherein the mechanical stirring means is contained in the closed type mixing chamber.

48. The device according to claim 47, wherein the stirring means occupies 10% to 99% of the volume of the closed type mixing chamber.

49. The device according to claim 47, wherein the mechanical stirring means comprises a stirrer axis and a stirring blade, in which the stirrer axis is positioned within the single outlet of the closed type mixing chamber.

50. The device according to claim 47, wherein the stirring means comprises a first and second stirring means oppositely arranged, such that the first stirring means is located near or at the top of the closed type mixing chamber and a second stirring means is located near or at the bottom of the closed type mixing chamber, wherein the each of the first and second stirring means comprises stirring blades disposed inside the closed type mixing chamber, said stirring blades having a magnetic coupling relationship with outer magnets disposed outside the closed type mixing chamber and the outer magnets being rotation-driven by motors connected to the outer magnets.

51. An organic compound in particulate form obtained by the process according to claim 35.

52. The organic compound according to claim 51, wherein the particulates have a D50 of less than 500 nm,

53. An active pharmaceutical ingredient comprising a precipitate obtained by the process according to claim 35.
